(19) United States
(12) Patent Application Publication
(10) Pub. No.: US 2023/0227909 A1
(43) Pub. Date: Jul. 20, 2023

(54) METHODS OF DETERMINING THE ETIOLOGY OF ACUTE ISCHEMIC STROKES

(71) Applicants: INSERM (INSTITUT NATIONAL DE A SANTÉ ET DE LA RECHERCHE MÉDICALE), Paris (FR); UNIVERSITÉ PARIS CITÉ, Paris (FR); FONDATION OPHTALMOLOGIQUE ADOLPHE DE ROTHSCHILD, Paris (FR); UNIVERSITÉ PARIS XIII PARIS-NORD, Villetaneuse (FR)

(72) Inventors: Mikhael MAZIGHI, Paris (FR); Jean-Philippe DESILLES, Paris (FR); Raphaël BLANC, Paris (FR); Véronique OLLIVIER, Paris (FR); Michel PIOTIN, Paris (FR); Lucas DI MEGLIO, Paris (FR); Benoit HO-TIN-NOE, Paris (FR); Mialitiana SOLO NOMENJANAHARY, Paris (FR)

(21) Appl. No.: 17/999,659

(22) PCT Filed: May 26, 2021
(86) PCT No.: PCT/EP2021/064009

§ 371 Date: Nov. 22, 2022

(30) Foreign Application Priority Data
May 27, 2020 (EP) 20305551.2

(51) Int. Cl. C12Q 1/6883

(52) U.S. Cl. CPC C12Q 1/6883; C12Q 2600/112; C12Q 2600/156

(57) ABSTRACT

Determining acute ischemic stroke (AIS) etiology is crucial for guidance of secondary prevention. Here, the inventors performed a correlation analysis between AIS etiology and AIS thrombus cellular composition and content, as assessed using quantitative biochemical assays. In particular, homogenates of 250 AIS patient thrombi were prepared by mechanical grinding. Platelet, red blood cell, and leukocyte content of AIS thrombi were estimated by quantification of glycoprotein (GP)VI, heme, and DNA in thrombus homogenates. AIS etiology was defined as cardioembolic, non-cardioembolic, or embolic stroke of undetermined source (ESUS), according to the TOAST classification. Cardioembolic thrombi were richer in DNA (35.8 vs 13.8 ng/mg, p<0.001) and poorer in GPVI (0.104 vs 0.117 ng/mg, p=0.045) than non- cardioembolic ones. The area under the receiver operating characteristic curve of DNA content to discriminate cardioembolic thrombi from non-cardioembolic was 0.72 (95% Cl, 0.63 to 0.81). With a threshold of 44.7 ng DNA/mg thrombus, 47% of thrombi from undetermined etiology would be classified as cardioembolic with a specificity of 90%. In conclusion, thrombus DNA content may provide an accurate biomarker for identification of cardioembolic thrombi in AIS patients with ESUS.

### FIELD OF THE INVENTION

The present invention is in the field of medicine and in particular cardiovascular diseases.

### BACKGROUND OF THE INVENTION

Acute ischemic stroke (AIS) can result from various mechanisms, such as large artery atherosclerosis or cardioembolism¹. Determining AIS etiology is crucial for optimal patient management. Stroke etiology is indeed a key factor for secondary prevention decisions. Yet, in 30 to 40% of AIS patients, a specific stroke etiology cannot be determined². In the case of AIS due lo large vessel occlusion (LVO), it has been proposed that thrombus composition could help determine thrombus origin. Although AIS thrombi causing LVO have been shown to share the same basic components and structure³, they are highly heterogeneous in that they contain highly variable amounts and proportions of red blood cells (RBCs)⁴, platelets⁵, leukocytes⁵, fibrin⁶, and von Willebrand factor⁴. This heterogeneity in thrombus composition has been suggested to reflect that in AIS etiology. Nevertheless, previous studies have reported conflicting results regarding possible correlations between thrombus composition and AIS etiology. The lack of consistency in conclusions on this issue might be related, at least in part, to the fact that the vast majority of studies on thrombus composition have been based on semiquantitative histological analyses using nonspecific staining methods of thrombus components⁴⁻⁷. In addition, considering the large inter- and/or intra-observer variability inherent to histological scoring strategies, such approaches may not allow for the development of accurate diagnostic tools.

### SUMMARY OF THE INVENTION

As defined by the claims, the present invention relates to methods of determining the etiology of acute ischemic strokes.

### DETAILED DESCRIPTION OF THE INVENTION

Determining acute ischemic stroke (AIS) etiology is crucial for guidance of secondary prevention. Previous studies have yielded inconsistent results regarding possible correlations between AIS etiology and thrombus composition, as assessed by semiquantitative histological analysis. Here, the inventors performed a correlation analysis between AIS etiology and AIS thrombus cellular composition and content, as assessed using quantitative biochemical assays. In particular, homogenates of 250 AIS patient thrombi were prepared by mechanical grinding. Platelet, red blood cell, and leukocyte content of AIS thrombi were estimated by quantification of glycoprotein (GP)VI, heme, and DNA in thrombus homogenates. AIS etiology was defined as cardioembolic, non-cardioembolic, or embolic stroke of undetermined source (ESUS), according to the TOAST classification. Cardioembolic thrombi were richer in DNA (35.8 vs 13.8 ng/mg, p<0.001) and poorer in GPVI (0.104 vs 0.117 ng/mg, p=0.045) than non-cardioembolic ones. The area under the receiver operating characteristic curve of DNA content to discriminate cardioembolic thrombi from non-cardioembolic was 0.72 (95% CI, 0.63 to 0.81). With a threshold of 44.7 ng DNA/mg thrombus, 47% of thrombi from undetermined etiology would be classified as cardioembolic with a specificity of 90%. In conclusion, thrombus DNA content may provide an accurate biomarker for identification of cardioembolic thrombi in AIS patients with ESUS.

Accordingly, the first object of the present invention relates to a method of determining the etiology of an acute ischemic stroke that occurred in a patient comprising quantifying the DNA content in the thrombus obtained from the patient wherein said level indicates a cardioembolic or a non-cardioembolic etiology.

As used herein, the term “stroke” has its general meaning in the art and refers to an episode of neurological dysfunction caused by focal cerebral, spinal, or retinal infarction (Easton et al., Stroke 2009, 40, 2276-2293). In particular, the term encompasses acute ischemic stroke (AIS), transient ischemic attack (TIA) and hemorrhagic stroke. Acute ischemic stroke can result from a variety of causes such as atherosclerosis of the cerebral circulation, occlusion of cerebral small vessels, and cardiac embolism. Cardiac embolism results from one of three mechanisms: blood stasis and thrombus formation in an enlarged (or affected by another structure alteration) left cardiac chamber (e.g., left ventricular aneurysm); release of material from an abnormal valvular surface (e.g., calcific degeneration); and abnormal passage from the venous to the arterial circulation (paradoxical embolism).

As used herein, the term “etiology” refers to the causes or origins, of diseases or abnormal physiological conditions. According to the present invention, the term “cardioembolic etiology” indicates that the stroke results from cardiac embolism. Oppositely, the term “a non-cardioembolic etiology” indicates that the stroke does not result from cardiac embolism.

The method of the present invention is particularly suitable for identifying “embolic stroke of undetermined source” or “ESUS” as defined in Hart RG, Diener HC, Coutts SB, Easton JD, Granger CB, O′Donnell MJ, Sacco RL, Connolly SJ Cryptogenic Stroke EIWG. Embolic strokes of undetermined source: the case for a new clinical construct. Lancet Neurol. 2014;13:429-438.

As used herein, the term “thrombus” or “blood clot” has its general meaning in the art and refers to a solid or semi-solid mass formed from the constituents of blood within the vascular system that is the product of blood coagulation. There are two components to a thrombus, aggregated platelets that form a platelet plug, and a mesh of cross-linked fibrin protein. The thrombus may obtained from the patient by any technique well known in the art. Typically, the thrombus is obtained from the patient during endovascular therapy (EVT) using a stent-retriever and/or a contact aspiration technique. Once obtained, the thrombus is of course, be subjected to a variety of well-known post-collection preparative and storage techniques such as described in the EXAMPLE for the in vitro purposes of the present invention.

As used herein, the term “DNA” has its general meaning in the art and refers to the deoxyribonucleic acid that is a molecule composed of two polynucleotide chains, each nucleotide is composed of one of four nitrogen-containing nucleobases (cytosine [C], guanine [G], adenine [A] or thymine [T]), a sugar called deoxyribose, and a phosphate group.

In some embodiments, DNA content may be quantified in the thrombus by any method well known in the art. Typically DNA content may be quantified by colorimetric or fluorometric assays which are typically performed by adding reagents to the sample obtained, which produces a color change, the degree of which correlates with the level of DNA. Other assays include hemagglutinin inhibition, complement fixation, and diffusion in agarose. Other assays involve RNA-DNA hybridization, RIA, and counter immunoelectrophoresis assays that allow quantification of nanogram amounts of DNA. With real-time PCR and PicoGreen doublestranded DNA quantification assays, picogram DNA content can be also quantified. Those skilled in the art will readily appreciate various methods to determine the DNA content of the thrombus; the methods suggested are merely for purposes of example.

In some embodiments, the higher is the DNA content, the higher is the probability that the patient had a cardioembolic stroke.

In some embodiments, the method of the present invention comprises the steps of i) quantifying the DNA content in the thrombus obtained from the patient ii) comparing the content quantified at step i) with a predetermined reference value and iii) concluding that the patient had a cardioembolic stroke when the content quantified at step i) is higher than the predetermined reference value or inversely concluding that the patient had a non-cardioembolic stroke when the content quantified at step i) is lower than the predetermined reference value.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the DNA content in the thrombus, one can use algorithmic analysis for the statistic treatment of the DNA content determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the present invention further comprises quantifying the GPVI content in the thrombus of the patient.

As used herein the term “GPVI” has its general meaning in the art and refers to platelet glycoprotein VI. Methods for quantifying GPVI content are well known in the art and typically and typically include immunoassays as described in the EXAMPLE.

In some embodiments, the DNA/GPVI ratio is calculated. In some embodiments, the higher is the DNA/GPVI ratio the higher is the probability that the patient had a cardioembolic stroke. In some embodiments, the method of the present invention comprises the steps of i) calculating the DNA/GPVI ratio ii) comparing the ratio calculated at step i) with a predetermined reference value and iii) concluding that the patient had a cardioembolic stroke when the ratio calculated at step i) is higher than the predetermined reference value or inversely concluding that the patient had a non-cardioembolic stroke when the ratio calculated at step i) is lower than the predetermined reference value.

The method is particularly suitable for determining whether the patient is eligible to a particular therapy, i.e. a secondary stroke prevention.

In particular, the method of the present invention is particularly suitable for determining whether the patient is eligible to an anticoagulant therapy. In particular, when it is concluded that the patient had a cardioembolic stroke then the patient is eligible with an anticoagulant therapy. As used herein, the term “anticoagulant” has its general meaning in the art and refers to a compound which is capable of preventing or inhibiting blood coagulation. Various compounds have been described as anticoagulants which affect one or more enzymes or auxiliary substances of the coagulation cascade. Coumarins, e.g., are plant-derived vitamin k antagonists which deplete the organism of the active form of vitamin K which is required as an auxiliary substance for thrombin and factors VII, IX and X activities. Typical coumarins include warfarin, acenocoumarol, phenprocoumon, atromentin, brodifacoum or phenindione. Heparins are highly sulfated glycosaminoglycanes and resemble another class of naturally occurring anticoagulants. They activate antithrombin which blocks the activity of thrombin and other enzymes of the coagulation cascade including factor Xa and, thereby, inhibit fibrin clot formation. Typically, low molecular weight heparin (LMWH) or unfractionated heparin (UFH) is used as heparin in anticoagulation therapy. Also heparanoids are used in anticoagulation therapy such as Danaparoid (also called Orgaran). In some embodiments, the anticoagulant is a factor Xa inhibitor. As used herein, the term “factor Xa inhibitor” refers to the ability of a compound to alter the function of factor Xa. A factor Xa inhibitor may block or reduce the activity of factor Xa by forming a reversible or irreversible covalent bond between the inhibitor and factor Xa or through formation of a noncovalently bound complex. In some embodiments, inhibition of factor Xa may be assessed using the method described in Wong et al, Journal of Thrombosis and Haemostasis 2008, 6(5), 820-829; Weitz et al, Thromb. Haemost. 2006, 96(3), 274-284; Turpie, A., Arterioscler Thromb Vasc Biol. 2007, 27, 1238-1247; Turpie, A., European Heart Journal 2007, 29, 155-165; and Jiang, et al., Thrombosis and Haemostasis 2009, 101(4), 780-782. Examples of factor Xa inhibitors include but are not limited to tamixaban, rivaroxaban, fondaparinux, and idraparinux. In some embodiments, the anticoagulant is thus selected from the group consisting of:
a direct thrombin inhibitor, such as dabigatran, hirudin, bivalirudin, lepirudin or argatroban,
a direct factor Xa inhibitor, such as rivaroxaban, apixaban, edoxaban, betrixaban, darexaban, letaxaban or eribaxaban,
a pentasaccharide, such as fondaparinux or idraparinux,
a low molecular weight heparins, such as nadroparin, tinzaparin, dalteparin, enoxaparin, bemiparin, reviparin, parnaparin or certoparin, unfractionated heparin,
a vitamin K antagonist, such as acenocoumarol, phenprocoumon, warfarin, atromentin or phenindione, and
an antiplatelet drug, such as an irreversible cyclooxygenase inhibitors (such as aspirin or a derivative thereof or triflusal), an ADP receptor inhibitor (such as clopidogrel, prasugrel, ticagrelor, ticlopedine, cangrelor or elinogrel), a phosphodiesterase inhibitor (such as cilostazol), a PAR-1 antagonist (such as voraxapar), a GPIIB/IIIa inhibitor (such as abciximab, eptifibatide, tirofiban, roxifiban or orbofiban), an adenosine reuptake inhibitor (such as dipyridamole), a thromboxane inhibitor (such as ifetroban or picotamide) or a thromboxane receptor antagonist (such as terutroban or picotamide).

When it is concluded that the patient has a non-cardioembolic stroke then the patient may eligible with a therapy that consists in administering the patient with diuretic or the combination of a diuretic and an ACE-inhibitor to lower the blood pressure of the patient, and/or with a statin therapy. As used herein, the term “diuretic” denotes any drug that elevates the rate of urination and thus provides a means of forced diuresis. There are several categories of diuretics. All diuretics increase the excretion of water from bodies, although each class does so in a distinct way. In some embodiments, the diuretic is selected from bumetamide, furosemide, ethacrynic acid, torsemide, azosemide, muzolimine, piretanide, tripamide and the like; thiazide and thiazide-like diuretics, such as bendroflumethiazide, benzthiazide, chlorothiazide, hydrochlorothiazide, hydro-flumethiazide, methylclothiazide, polythiazide, trichlormethiazide, chlorthalidone, indapamide, metolazone and quinethazone; and analogs and functional derivatives of such compounds. The term “ACE inhibitor” is synonymous with the term ACE-I and describes an angiotensin converting enzyme inhibitor, i.e. an active substance acting mainly by inhibiting the synthesis of angiotensin H and by blocking the degradation of bradykinin. Examples of ACE-inhibitors include but are not limited to benazepril, captopril, cilazapril, enalapril, fosinopril, lisinopril, moexipril, perindopril, quinapril, ramipril, spirapril, and trandolapril. As used herein, the term “statin” designates at least one HMG-CoA reductase inhibitor. Preferably the statin is at least one ring-opened 7-substituted-3,5-dihydroxyheptanoic acid or ring-opened 7-substituted-3,5-dihydroxyheptenoic acid. The statin is preferably selected from the group consisting of lovastatin, mevastatin, simvastatin, pravastatin, atorvastatin, cerivastatin, itavastatin, fluvastatin, pitavastatin, rosuvastatin, and salts thereof.

The invention will be further illustrated by the following figures and examples. However, these examples and figures should not be interpreted in any way as limiting the scope of the present invention.

### FIGURES

FIG. 1. Distribution of biochemical features of AIS thrombi according to etiology. (A-D) Boxes show the 25th, 50th, and 75th, and whiskers indicate values outside the lower and upper quartile with a length equal to 1.5 interquartile range; diamond indicates the mean values. P-values for global comparison (one-way ANOVA) are reported after a log-transformation for DNA, and ratio DNA/GPVI; * indicated P-values <0.05 for post-hoc pairwise comparison between cardioembolic stroke and each other stroke subgroups (adjusted for multiple comparison using Bonferroni correction).

FIG. 2. Receiver operating characteristic (ROC) curve for differentiation of cardioembolic and non-cardioembolic strokes according to DNA and GPVI thrombus content, and to the DNA/GPVI thrombus content ratio.

### EXAMPLE

### Methods

### Standard Protocol Approvals, Registrations, and Patient Consents

Thrombi were collected in two centers at the end of endovascular therapy (EVT). The EVT procedure was chosen at the interventionalist’s discretion, using a stent-retriever and/or a contact aspiration technique. AIS etiology was classified as described¹ and determined based on cerebral magnetic resonance imaging (MRI), computed tomography or MRI angiography, transcranial and extracranial duplex sonography, coagulation tests, 1 to 3 days electrocardiography recording, and transthoracic and/or transesophageal echocardiography. Patient data were collected prospectively using a standardized questionnaire (Endovascular Treatment in Ischemic Stroke -ETIS- registry NCT03776877). All patients were provided with a written explanation of the study. The patients or their representatives were given the opportunity to refuse participation. The local Ethics Committee approved this research protocol (CPP Nord Ouest II, ID-RCB number: 2017-A01039-44).

### Preparation of Thrombus Homogenates

Thrombus homogenates were prepared with stainless steel beads (5 mm, Qiagen, 69989) in cold PBS (30 µL/mg thrombus) supplemented with protease inhibitor (1%, Sigma, P8340), using a tissue lyser (25 Hz, 4 minutes, TissueLyser II, Qiagen). Thrombi not completely grinded went through a second passage in the tissue lyser. The thrombus homogenates were then recovered after centifugation (14 000 g x 20 minutes, 4° C.) to eliminate non-soluble debris. Homogenates of initially cut thrombi were pooled before analysis.

### Quantification of Red Blood Cell and DNA

RBC content was estimated by measurement of heme concentration in thrombus homogenates using a formic acid-based colorimetric assay, as described previously⁸. DNA was quantified using the Molecular Probes Quant iT Picogreen dsDNA Assay kit (Life Technologies).

### Quantification of Platelet Content

Soluble GPVI levels were measured by immunoassay according to the following protocol. Ninety-six wells standard binding plate from MesoScale Discovery (MSD, Rockville, MD) were coated overnight at 4° C. with 2 µg/mL sheep anti human GPVI polyclonal antibody (Bio Techne, France, AF3627). After 1 hour of incubation at room temperature with 5% MSD Blocker A (R93AA-1) and 3 washes with 150 µL PBS / 0.05% Tween (PBST), 25 µL of thrombus homogenate or standard were added and the plate was incubated for 1 hour at room temperature, 500 rpm. Standard curve was obtained with Recombinant Human GPVI protein (Bio techne, France, 3627-GP, 0.097-25 ng/ml). After 3 PBST washes, 25 µL of biotinylated sheep anti-human GPVI antibody (Bio Techne, France, BAF3627, 0.5 µg/mL in 1% MSD Blocker A) was added to each well and the plate was incubated 1 hour at room temperature. Finally, 25 uL of streptavidin Sulfo-TAG/well was added after 3 PBST washes and the plate was incubated 1 hour at room temperature. A MesoScale Quickplex Plate Scanner was used of quantification.

### Statistical Analysis

Categorical variables were expressed as frequencies and percentages. Quantitative variables were expressed as mean (standard deviation, SD), or median (interquartile range, IQR) for non-normal distribution. Normality of distributions was assessed graphically and by using the Shapiro-Wilk test. We compared the different proportions of components of thrombi (heme, DNA, platelet, and DNA/platelet ratio) between the 3 AIS etiology subgroups (cardioembolic, non cardioembolic and ESUS) using one-way analysis of variance (ANOVA); post-hoc pairwise comparisons were done using linear contrast after Bonferroni correction. Primary comparison covered the overall study sample and was further performed according to use of IV alteplase prior to EVT. For thrombus content which were significant between the two group of interest (cardioembolic vs. non cardioembolic), we assessed the performance of thrombus content to determine cardioembolic from noncardioembolic etiology by calculating the area under the ROC curves (AUCs) and their 95% confidence intervals (CIs). From the ROC curves, we determined the optimal threshold value by maximizing the Youden index as well as the threshold values to reach a sensitivity and specificity of 0.90, respectively. We applied these threshold value in the cryptogenic patients. Statistical testing was conducted at the two-tailed α-level of 0.05. Data were analyzed using the SAS software version 9.4 (SAS Institute, Cary, NC).

### Results

From June 2016 to November 2018, a total of 1209 consecutive AIS patients with LVO were treated by EVT in our institutions. Thrombi from 250 of these patients selected randomly were homogenized and analyzed for RBC, platelet, and leukocyte content, as estimated by quantification of heme, GPVI, and DNA, respectively. Patient and treatment characteristics of the study sample are reported in Table 1. Stroke etiology was cardioembolic in 142 (56.8%) patients, non-cardioembolic in 33 patients (13.2%), and undetermined in 75 patients (30.0%).

### Thrombus Cellular Content and AIS Etiology

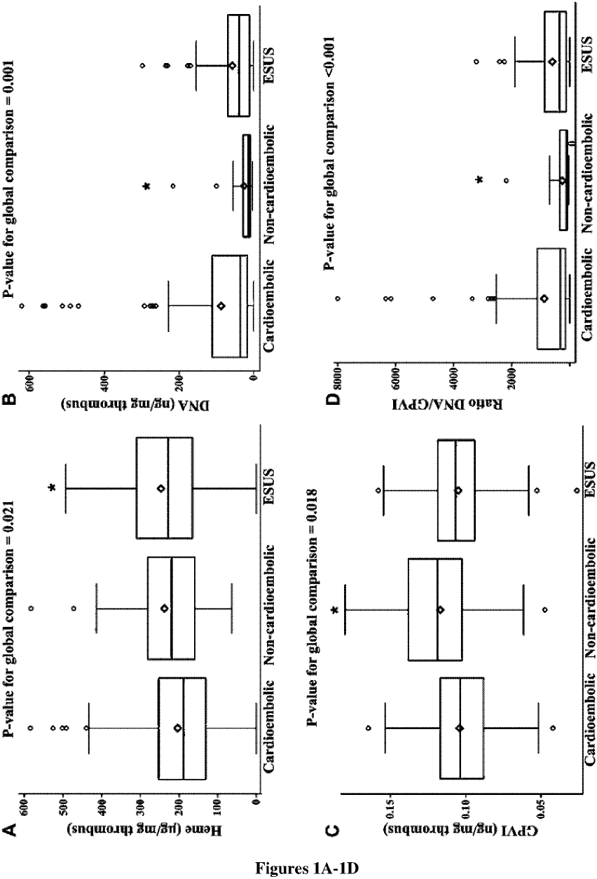

There was no significant difference in the heme content between thrombi from cardioembolic and non-cardioembolic origin (FIG. 1A).

Non-cardioembolic thrombi had reduced DNA content, and increased GPVI content as compared to cardioembolic thrombi (FIGS. 1B and C). As a consequence, the DNA/GPVI ratio (FIG. 1D) was higher in cardioembolic thrombi than in non-cardioembolic ones (median IQR : 322 (151 to 1132) vs 266 (151 to 1132), p<0.001). Together, these results indicate that cardioembolic thrombi contain significantly more leukocytes and less platelets than non-cardioembolic ones.

Thrombi from undetermined etiology had increased heme content compared to cardioembolic thrombi (FIG. 1A), but showed no significant differences in DNA or platelet content as compared to either of the other groups of thrombi (FIGS. 1B-D).

### Thrombus DNA Content to Discriminate Cardioembolic Versus Non-Cardioembolic AIS

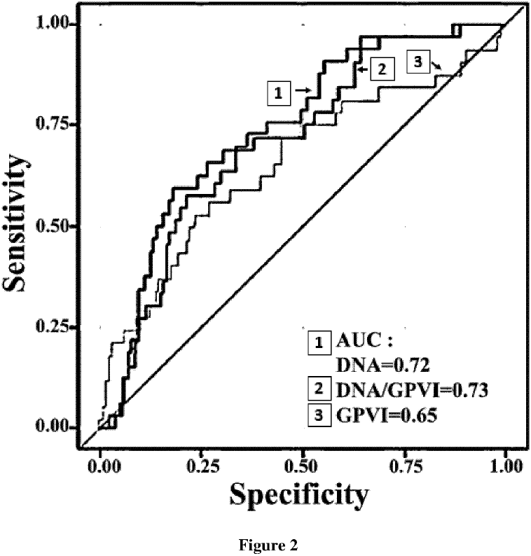

The area under the receiver operating characteristic curve (AUC) for thrombus DNA content used for differentiating thrombi of cardioembolic and non-cardioembolic origins was of 0.72 (95% CI, 0.63 to 0.81). A similar AUC value was obtained for the DNA/GPVI ratio (FIG. 2 and Table 2). These data suggest that both thrombus DNA content and DNA/GPVI ratio hold potential usefulness for identification of cardioembolic thrombi. In contrast, the AUC for the GPVI thrombus content was of 0.65 (95% CI, 0.54 to 0.77) (FIG. 2 and Table 2), indicating a poor diagnostic potential. The specificity and sensitivity of thrombus DNA content for discriminating cardioembolic thrombi from non-cardioembolic thrombi was calculated for various thresholds of DNA thrombus content (Table 2). For a threshold of 44.7 ng DNA/mg thrombus, nearly 50% of ESUS thrombi would be classified as cardioembolic with a specificity of 90%.

### Discussion

In the present study conducted on 250 AIS thrombi responsible for LVO, we have explored possible relationships between AIS etiology and thrombus cell composition. In order to avoid the inherent limitations of semi-quantitative immunohistological methods⁷, we have analyzed cell composition using quantitative assays for markers of RBCs, platelets, and leukocytes. Our results show that cardioembolic thrombi are richer in DNA and poorer in platelets compared to non-cardioembolic thrombi. From a pathophysiological perspective, the increased DNA content of thrombi from cardioembolic origin suggests a more prominent role of leukocytes in the formation of those thrombi. Leukocytes, especially neutrophils, are indeed the primary source of DNA in blood and are now widely recognized as active players of thrombosis^{9,10}. Interestingly, previous studies have shown that elevated neutrophil-lymphocyte ratios in patients with nonvalvular atrial fibrillation were independently associated with the presence of left atrial thrombus¹¹, as well as with an increased risk of thromboembolic stroke¹². Also consistent with our results, patients with cardioembolic stroke were reported to have increased plasma cell-free DNA levels compared to stroke patients of other etiologies¹³.

The increased DNA content of cardioembolic thrombi might also account for their previously reported higher leukocyte and neutrophil extracellular traps (NETs) content compared to thrombi of other origins¹⁴. Additionally, the high proportion of DNA content found in cardioembolic thrombi and the pivotal role of neutrophils and NETs in thrombosis give additional arguments for a potential benefit of DNAse 1 in AIS treatment^{14,15}.

Importantly, our results indicate that both the thrombus DNA content and the thrombus DNA/GPVI ratio could provide biomarkers for identification of cardioembolic thrombi among thrombi of undetermined origin. In fact, specificity/selectivity calculations revealed that, by adjusting the DNA thrombus content threshold, one could classify nearly 50% of ESUS thrombi as cardioembolic with a specificity of 90%. Considering that ESUS represents 20-25% of all AIS, there is a clear interest in developing new diagnostic tools to better identify ESUS patient subgroups. A recent major secondary prevention trial found no superiority of rivaroxaban over aspirin for prevention of recurrent stroke in the overall ESUS patient population¹⁶. Identifying the subgroup of ESUS patients requiring more active cardiac screening and which could benefit from anticoagulant therapy could help to both improve patient management and refine secondary prevention studies.

In addition to be inexpensive, thrombus homogenization as performed in our study requires only moderate skills and is fairly easily feasible with common laboratory and hospital equipment, and so is the subsequent measurement of DNA in thrombus homogenates. The main limitation of this method based on mechanical grinding of AIS thrombi is that non-soluble components such as fibrin could not be directly quantified.

To date, and to our knowledge, it is the largest study on thrombus composition based on biochemical quantitative analysis of their cellular content. Our results provide a potential basis for the development of new tools and strategies for identification of ESUS patient subgroups and improved secondary prevention.

### Tables

TABLE 1

Patients and treatment characteristics, in overall and according to suspected acute ischemic stroke etiology

| Characteristics | Suspected AIS etiology | | | |
|---|---|---|---|---|
| | Overall | Cardioembolic | Non-cardioembolic | ESUS |
| Number of patients | 250 | 142 | 33 | 75 |
| Demographics | | | | |
| Age, years, mean (SD) | 70.1 (15.5) | 74.4 (14.6) | 62.2 (12.9) | 65.3 (15.5) |
| Men, n (%) | 129/250 (51.6) | 66/142 (46.5) | 24/33 (72.7) | 39/75 (52.0) |
| Medical history | | | | |
| Hypertension | 144/247 (58.3) | 92/141 (65.2) | 14/32 (43.8) | 38/74 (51.4) |
| Diabetes | 42/248 (16.9) | 25/142 (17.6) | 6/32 (18.8) | 11/74 (14.9) |
| Hypercholesterolemia | 79/247 (32.0) | 52/141 (36.9) | 9/32 (28.1) | 18/74 (24.3) |
| Current smoking | 50/238 (21.0) | 22/134 (16.4) | 7/32 (21.9) | 21/72 (29.2) |
| Coronary artery disease | 32/245 (13.1) | 21/139 (15.1) | 3/33 (9.1) | 8/73 (11.0) |
| Previous stroke or TIA | 36/246 (14.2) | 23/139 (16.5) | 5/33 (15.2) | 7/74 (9.5) |
| Previous antithrombotic medications | 103/244 (42.2) | 81/140 (57.9) | 7/31 (22.6) | 15/73 (20.5) |
| Antiplatelet | 47/244 (19.3) | 29/140 (20.7) | 5/31 (16.1) | 13/73 (17.8) |
| Anticoagulant | 48/244 (19.7) | 44/140 (31.4) | 2/31 (6.5) | 2/73 (2.7) |

Current stroke event

| | | | | |
|---|---|---|---|---|
| NIHSS score, median (IQR)^{a} | 17 (12 to 20) | 18 (14 to 21) | 16 (9 to 19) | 16 (12 to 20) |
| Pre-stroke mRS≥1 | 23/248 (9.2) | 30/141 (21.3) | 5/33 (15.2) | 8/74 (10.8) |
| ASPECTS, median (IQR)^{b} | 7 (5 to 8) | 7 (6 to 8) | 6 (5 to 8) | 6 (5 to 8) |
| Site of occlusion | | | | |
| M1-MCA | 134/246 (54.5) | 80/139 (57.6) | 7/33 (21.2) | 47/74 (63.5) |
| M2-MCA | 20/246 (8.1) | 14/139 (10.1) | 0 (0.0) | 6/74 (8.1) |
| Intracranial ICA or tandem | 53/246 (21.5) | 28/139 (20.1) | 7/33 (21.2) | 18/74 (24.3) |
| Tandem | 19/246 (7.7) | 5/139 (3.6) | 14/33 (42.4) | 0 (0.0) |
| extracranial ICA | 6/246 (2.4) | 4/139 (2.9) | 1/33 (3.0) | 1/74 (1.4) |
| Vertebro-Basilar | 12/246 (4.9) | 6/139 (4.3) | 4/33 (12.1) | 2/74 (2.7) |
| Others | 2/246 (0.8) | 2/139 (1.4) | 0 (0.0) | 0 (0.0) |

Treatment characteristics

| | | | | |
|---|---|---|---|---|
| Intravenous Alteplase | 131/250 (52.4) | 62/142 (43.7) | 20/33 (60.6) | 49/75 (65.3) |
| General anesthesia | 38/242 (15.7) | 22/138 (15.9) | 7/30 (23.3) | 9/74 (12.2) |
| Onset to groin puncture time, min, median (IQR)^{c} | 240 (186 to 286) | 222 (170 to 279) | 262 (217 to 308) | 250 (205 to 295) |

Values expressed as no/total no. (%) unless otherwise indicated. ^{a}3 missing data (2 in cardioembolic group and 1 in Non-cardioembolic group) ^{b}18 missing data (12 in cardioembolic group, 1 in Non-cardioembolic group and 5 in Cryptogenic group) ^{c}7 missing data (4 in cardioembolic group, 1 in Non-cardioembolic group and 2 in Cryptogenic group).
Abbreviations: ASPECTS= Alberta stroke program early computed tomography score; ICA=internal carotid artery; IQR=interquartile range; MCA=middle cerebral artery; NIHSS=National Institutes of Health Stroke Scale; rt-PA=recombinant tissue plasminogen activator; TIA=transient ischemic attack; mRS=modified Rankin scale, SD=standard deviation.

TABLE 2

Accuracy of thrombus cell marker content for identification of cardioembolic thrombi

| | AUC (95%CI) | Threshold | Sensitivity (95%CI) | Specificity (95%CI) | % of patients with ESUS |
|---|---|---|---|---|---|
| DNA | 0.72 (0.63 to 0.81) | >22.4¹ | 66.0 (57.5 to 73.7) | 69.7 (51.3 to 84.4) | 62.5 |
| | | >8.9 | 90.0 | 27.3 (13.3 to 45.5) | 84.7 |
| | | >44.7 | 44.0 (35.6 to 52.3) | 90.0 | 47.2 |
| GPVI | 0.65 (0.54 to 0.77) | <11.5¹ | 56.2 (37.7 to 73.6) | 89.2 (82.6 to 94.0) | 71.9 |
| | | <13.4 | 90.0 | 28.1 (13.7 to 46.7) | 90.6 |
| | | <7.7 | 10.0 (5.4 to 16.5) | 90.0 | 12.5 |
| DNA/GPVI | 0.73 (0.63 to 0.82) | >161¹ | 72.9 (64.3 to 80.3) | 65.6 (46.8 to 81.4) | 65.6 |
| | | >81 | 90.0 | 34.4 (18.6 to 53.2) | 81.2 |
| | | >614 | 36.4 (28.1 to 45.4) | 90.0 | 31.2 |

¹ cut-value who maximize the Youden index.
Abbreviations: AUC=area under the Receiver Operating Curve; CI=confidence interval.

### REFERENCES

Throughout this application, various references describe the state of the art to which this invention pertains. The disclosures of these references are hereby incorporated by reference into the present disclosure.
1. Adams HP, Bendixen BH, Kappelle LJ, Biller J, Love BB, Gordon DL, et al. Classification of subtype of acute ischemic stroke. Definitions for use in a multicenter clinical trial. TOAST. Trial of Org 10172 in Acute Stroke Treatment. Stroke. 1993;24:35-41.
2. Yaghi S, Bernstein RA, Passman R, Okin PM, Furie KL. Cryptogenic Stroke: Research and Practice. Circ. Res. 2017;120:527-540.
3. Di Meglio L, Desilles J-P, Ollivier V, Nomenjanahary MS, Di Meglio S, Deschildre C, et al. Acute ischemic stroke thrombi have an outer shell that impairs fibrinolysis. Neurology. 2019;
4. Denorme F, Langhauser F, Desender L, Vandenbulcke A, Rottensteiner H, Plaimauer B, et al. ADAMTS13-mediated thrombolysis of t-PA-resistant occlusions in ischemic stroke in mice. Blood. 2016;127:2337-2345.
5. Sporns PB, Hanning U, Schwindt W, Velasco A, Minnerup J, Zoubi T, et al. Ischemic Stroke: What Does the Histological Composition Tell Us about the Origin of the Thrombus? Stroke. 2017;48:2206-2210.
6. Hashimoto T, Hayakawa M, Funatsu N, Yamagami H, Satow T, Takahashi JC, et al. Histopathologic Analysis of Retrieved Thrombi Associated with Successful Reperfusion after Acute Stroke Thrombectomy. Stroke. 2016;47:3035-3037.
7. De Meyer SF, Andersson T, Baxter B, Bendszus M, Brouwer P, Brinjikji W, et al. Analyses of thrombi in acute ischemic stroke: A consensus statement on current knowledge and future directions. International Journal of Stroke. 2017;1747493017709671.
8. Delbosc S, Bayles RG, Laschet J, Ollivier V, Ho-Tin-Noe B, Touat Z, et al. Erythrocyte Efferocytosis by the Arterial Wall Promotes Oxidation in Early-Stage Atheroma in Humans. Frontiers in Cardiovascular Medicine [Internet]. 2017 [cited 2019 Mar 1];4. Available from: http://journal.frontiersin.org/article/10.3389/fcvm.2017.00043/full
9. Martinod K, Wagner DD. Thrombosis: tangled up in NETs. Blood. 2014;123:2768-2776.
10. Fuchs TA, Brill A, Wagner DD. Neutrophil extracellular trap (NET) impact on deep vein thrombosis. Arterioscler. Thromb. Vasc. Biol. 2012;32: 1777-1783.
11. Yalcin M, Aparci M, Uz O, Isilak Z, Balta S, Dogan M, et al. Neutrophil-lymphocyte ratio may predict left atrial thrombus in patients with nonvalvular atrial fibrillation. Clin. Appl. Thromb. Hemost. 2015;21:166-171.
12. Ertaş G, Sönmez O, Turfan M, Kul S, Erdoǧan E, Tasal A, et al. Neutrophil/lymphocyte ratio is associated with thromboembolic stroke in patients with nonvalvular atrial fibrillation. J. Neurol. Sci. 2013;324:49-52.
13. Vallés J, Lago A, Santos MT, Latorre AM, Tembl JI, Salom JB, et al. Neutrophil extracellular traps are increased in patients with acute ischemic stroke: prognostic significance. Thromb. Haemost. 2017;117:1919-1929.
14. Laridan E, Denorme F, Desender L, François O, Andersson T, Deckmyn H, et al. Neutrophil extracellular traps in ischemic stroke thrombi. Ann Neurol. 2017;82:223-232.
15. Ducroux C, Di Meglio L, Loyau S, Delbosc S, Boisseau W, Deschildre C, et al. Thrombus Neutrophil Extracellular Traps Content Impair tPA-Induced Thrombolysis in Acute Ischemic Stroke. Stroke. 2018;49:754-757.
16. Hart RG, Sharma M, Mundl H, Kasner SE, Bangdiwala SI, Berkowitz SD, et al. Rivaroxaban for Stroke Prevention after Embolic Stroke of Undetermined Source. N. Engl. J. Med. 2018;378:2191-2201.

1. A method of determining the etiology of an acute ischemic stroke that occurred in a patient and treating the patient comprising
quantifying the DNA content in a thrombus obtained from the patient and
i) determining that the DNA content is higher than a predetermined reference value and treating the patient for cardioembolic stroke by administering an anticoagulant to the patient;
or
ii) determining that the DNA content is lower than the predetermined reference value and treating the patient for non-cardioembolic stroke by administering a diuretic, a combination of a diuretic and an ACE-inhibitor and/or a statin therapy to the patient.

2. The method of claim 1 wherein the cardioembolic stroke is of undetermined source.

3. (canceled)

4. (canceled)

5. The method of claim 1 further comprising quantifying the GPVI content in the thrombus of the patient.

6. The method of claim 5 wherein a DNA/GPVI ratio is calculated.

7. (canceled)

8. A method of determining the etiology of an acute ischemic stroke that occurred in a patient and treating the patient comprising
quantifying the DNA content in a thrombus obtained from the patient,
quantifying the GPVI content in the thrombus,
calculating a DNA/GPVI ratio and
i) determining that the DNA/GPVI ratio is higher than a predetermined reference value and treating the patient for cardioembolic stroke by administering an anticoagulant to the patient;
or
ii) determining that the DNA/GPVI ratio is lower than the predetermined reference value and treating the patient for non-cardioembolic stroke by administering n diuretic, a combination of a diuretic and an ACE-inhibitor and/or a statin therapy to the patient.

9. (canceled)

10. The method of claim 1, wherein the anticoagulant is selected from the group consisting of:
a direct thrombin inhibitor,
a direct factor Xa inhibitor,
a pentasaccharide,
a low molecular weight heparin,
a vitamin K antagonist, and
an antiplatelet drug.

11. (canceled)

12. The method of claim 10, wherein
the direct thrombin inhibitor is dabigatran, hirudin, bivalirudin, lepirudin or argatroban,
the direct factor Xa inhibitor is rivaroxaban, apixaban, edoxaban, betrixaban, darexaban, letaxaban or eribaxaban,
the pentasaccharide is fondaparinux or idraparinux,
the low molecular weight heparin is nadroparin, tinzaparin, dalteparin, enoxaparin, bemiparin, reviparin, parnaparin or certoparin or unfractionated heparin,
the vitamin K antagonist is acenocoumarol, phenprocoumon, warfarin, atromentin or phenindione, and
the antiplatelet drug is an irreversible cyclooxygenase inhibitor, an ADP receptor inhibitor, a phosphodiesterase inhibitor, a PAR-1 antagonist, a GPIIB/IIIa inhibitor, an adenosine reuptake inhibitor, a thromboxane inhibitor or a thromboxane receptor antagonist.

13. The method of claim 12, wherein
the irreversible cyclooxygenase inhibitors is aspirin or a derivative thereof or triflusal,
the ADP receptor inhibitor is clopidogrel, prasugrel, ticagrelor, ticlopedine, cangrelor or elinogrel,
the phosphodiesterase inhibitor is cilostazol,
the PAR-1 antagonist is voraxapar,
the GPIIB/IIIa inhibitor is abciximab, eptifibatide, tirofiban, roxifiban or orbofiban,
the adenosine reuptake inhibitor is dipyridamole,
the thromboxane inhibitor is ifetroban or picotamide, and
the thromboxane receptor antagonist is terutroban or picotamide.

14. The method of claim 8, wherein the anticoagulant is selected from the group consisting of:
a direct thrombin inhibitor,
a direct factor Xa inhibitor,
a pentasaccharide,
a low molecular weight heparins,
a vitamin K antagonist, and
an antiplatelet drug.

15. The method of claim 14, wherein
the direct thrombin inhibitor is dabigatran, hirudin, bivalirudin, lepirudin or argatroban,
the direct factor Xa inhibitor is rivaroxaban, apixaban, edoxaban, betrixaban, darexaban, letaxaban or eribaxaban,
the pentasaccharide is fondaparinux or idraparinux,
the low molecular weight heparin is nadroparin, tinzaparin, dalteparin, enoxaparin, bemiparin, reviparin, parnaparin or certoparin or unfractionated heparin,
the vitamin K antagonist is acenocoumarol, phenprocoumon, warfarin, atromentin or phenindione, and
the antiplatelet drug is an irreversible cyclooxygenase inhibitor, an ADP receptor inhibitor, a phosphodiesterase inhibitor, a PAR-1 antagonist, a GPIIB/IIIa inhibitor, an adenosine reuptake inhibitor, a thromboxane inhibitor or a thromboxane receptor antagonist.

16. The method of claim 15, wherein
the irreversible cyclooxygenase inhibitors is aspirin or a derivative thereof or triflusal,
the ADP receptor inhibitor is clopidogrel, prasugrel, ticagrelor, ticlopedine, cangrelor or elinogrel,
the phosphodiesterase inhibitor is cilostazol,
the PAR-1 antagonist is voraxapar,
the GPIIB/IIIa inhibitor is abciximab, eptifibatide, tirofiban, roxifiban or orbofiban,
the adenosine reuptake inhibitor is dipyridamole,
the thromboxane inhibitor is ifetroban or picotamide, and
the thromboxane receptor antagonist is terutroban or picotamide.
